(19) United States
(12) Patent Application Publication
(10) Pub. No.: US 2019/0060414 A1
(43) Pub. Date: Feb. 28, 2019

(54) METHODS OF TREATING EPILEPSY, SEIZURE DISORDERS AND SUDDEN UNEXPECTED DEATH IN EPILEPSY

(71) Applicant: HealthPartners Institute, Bloomington, MN (US)

(72) Inventors: William H. Frey, II, St. Paul, MN (US); Leah R. Bresin Hanson, Vadnais Heights, MN (US)

(21) Appl. No.: 16/111,500

(22) Filed: Aug. 24, 2018

Related U.S. Application Data
(60) Provisional application No. 62/550,857, filed on Aug. 28, 2017.

(51) Int. Cl. A61K 38/28; A61K 9/00; A61P 25/08

(52) U.S. Cl. CPC A61K 38/28; A61P 25/08; A61K 9/0043

(57) ABSTRACT

The present system is directed in several embodiments to a method of administration of a therapeutic composition for treatment of seizures resulting from epilepsy and other seizure disorders. The method includes administering one or more therapeutic compositions comprising an effective amount of insulin directly to the subject patient's CNS, with no to minimal systemic exposure. Preferably, this method comprises administration of an effective amount of insulin to the upper third of a patient's nasal cavity, thereby bypassing the patient's blood-brain barrier and delivering the therapeutic composition directly to the patient's central nervous system.

### CROSS-REFERENCE TO RELATED APPLICATIONS

This application claims priority to App. Ser. No. 62/550857, entitled “Methods of Treating Epilepsy, Seizure Disorders and Sudden Unexpected Death in Epilepsy,” filed Aug. 28, 2017, the entire contents of which is hereby incorporated by reference in its entirety

### FEDERAL FUNDING

None.

### BACKGROUND OF THE INVENTION

### Field of the Invention

The present invention is directed to methods of treating patients diagnosed with epilepsy or seizure disorders and includes treatment that reduces the risk of sudden unexpected death in patients with epilepsy. More specifically, the invention is directed to administration of intranasal insulin to the upper third of the patient's nasal cavity to prevent seizures or minimize the effects of seizures in patients with epilepsy or seizure disorders.

### Description of the Related Art

Delivery of the agent and/or composition to the upper one third of the patient's nasal cavity is a means of bypassing the blood brain barrier (“BBB”) to administer therapeutic compounds and/or agents directly to the CNS. This basic methodology is discussed and described in U.S. Pat. No. 5,624,898 to Frey II entitled Method for Administering Neurologic Agents to the Brain, as well as in U.S. Pat. No. 6,313,093 to Frey II, the entire contents of each of which are hereby incorporated by reference. This administration technique is a vast improvement over systemic administration methods such as intravenous and oral administration of drugs, which generally cannot cross the BBB to reach their targets within the CNS. In addition, Frey's intranasal method is a significant improvement over the general inhalation methods, which target the lower two-thirds of the patient's nasal cavity. Both the systemic and general intranasal method targeting the lower two-thirds of the nasal cavity result in a very large, unwanted and potentially dangerous systemic exposure to the administered drug or therapeutic agent(s). The present invention addresses, inter alia, this general intranasal problem as well as ensures that the patient's non-CNS, systemic disease and/or condition is protected from exposure to the therapeutic agent administered to the upper third of the nasal cavity, and potential harm therefrom.

General inhalation or intranasal methods target the lower two-thirds of the nasal cavity delivered by, e.g., nasal spray bottles, on the other hand, result in a large amount of systemic absorption and exposure, with a very small amount of the administered compound, i.e., less than 5%, making the tortuous journey around the turbinates to the upper third of the nasal cavity and still less compound than that very small amount further bypassing the BBB to actually reach the CNS.

Delivery and administration to the upper third of the nasal cavity, is very effective in administering the subject compounds or agents to the desired target, i.e., the CNS, without significant systemic exposure, though some systemic exposure does occur as is further discussed below.

Unwanted systemic exposure of therapeutics used to treat CNS diseases creates several serious problems. The systemic metabolism greatly reduces the bioavailability of any agent and/or compound exposed to the non-CNS system. This reduction of bioavailability is increased by unwanted plasma protein binding of the agent and/or compound. As a result, only a small amount of the active therapeutic agent and/or compound actually reaches the CNS. Because of these, inter alia, issues, the actual dose that must be administered in order to achieve a therapeutic dose in the targeted CNS is far larger than the therapeutic dosing. As a consequence, a relatively large concentration of the agent(s) and/or compounds(s) is in the system and will affect non-target systemic organs and systems. This can create unwanted and often dangerous side effects on these non-target organs and systems, particularly in the specific case of patient's having a systemic, non-CNS disorder or condition that contraindicates the systemic use or exposure of the therapeutic agent(s) needed to treat a CNS-related disorder or condition.

We have addressed the efficiency needs in patent application Ser. No. 12/134,385 to Frey II, et al., entitled “Pharmaceutical Compositions and Methods for Enhancing Targeting of Therapeutic Compounds to the Central Nervous System, the entire contents of which are hereby incorporated by reference, and wherein a vasoconstrictor is administered to the patient's nasal cavity either just prior to, or in combination with, administration of at least one therapeutic agent and/or pharmaceutical composition(s) comprising a therapeutic compound(s) and/or agent(s). The efficiency of the direct administration of the pharmaceutical compound to the CNS, with concomitant reduction of systemic exposure of the pharmaceutical compound is remarkable.

Moreover, we provide disclosure of the following patents and applications, each of which are commonly assigned with the present application and incorporated herein in their entirety for disclosure of, inter alia, the various diseases, conditions or disorders of the CNS relating herein to the first disease or condition of the present invention, as well as various compounds and/or therapeutic agents for treating same by application to the upper third of the nasal cavity, thereby bypassing the blood-brain barrier, with subsequent direct delivery of an effective amount of the compounds and/or agents to the CNS:

U.S. Pat. No. 7,776,312 Method of treating Alzheimer's disease comprising administering deferoxamine (DFO) to the upper one-third of the nasal cavity;

U.S. Pat. No. 7,618,615 Methods for providing neuroprotection for the animal central nervous system against neurodegeneration caused by ischemia;

U.S. Pat. No. 7,084,126 Methods and compositions for enhancing cellular function through protection of tissue components;

U.S. Pat. No. 6,313,093 Method for Administering Insulin to the Brain;

U.S. Pat Application 20100061959 Methods for Providing Neuroprotection for the Animal Central Nervous System Against the Effects of lschemia, Neurodegeneration, Trauma, and Metal Poisoning;

U.S. Patent Application 20110311654 Methods and Pharmaceutical Compositions for Treating the Animal Central Nervous System for Psychiatric Disorders.

Patients with epilepsy or seizure disorders that can include partial seizures, generalized seizures, febrile seizures, etc. generally have the ability to sense an impending seizure. Therefore, patients with epilepsy or recurring seizures could be treated regularly with intranasal insulin according to the present invention to reduce their risk for seizures or to reduce the frequency of seizures or the length of their seizures. It would be highly advantageous to provide a treatment method that may prevent the impending sensed seizure altogether or to minimize the effects of a seizure before the seizure begins and/or during the seizure itself. It is known that patients with epilepsy display a shortage in brain glucose consumption and that induction of chronic inhibition in glucose consumption in the brains of healthy rats can result in epilepsy. Thus, brain hypometabolism may not be a side effect of epilepsy, but may instead be a primary initiating factor in acquiring epilepsy.

It is known that patients who have seizures are able to generally sense that a seizure is impending. In some cases, there is a warning period of a reasonable time length, in other cases the warning period is quite short. The following discussion summarizes the four basic phases or stages of seizures.

A seizure often has four distinct phases: Prodromal Symptoms, Auras, Ictal and Postictal Stages.

The first phase—the prodromal stage—involves mostly emotional signals that, when sensed by a patient, may serve as an indication to take one or more doses of medication to prevent and/or minimize the impending symptoms.

In the aura stage, alterations in activity, emotions, hearing, smell, taste, visual perception are involved. The sensations within the aura stage may also serve as an indication to take one or more doses of medication to prevent and/or minimize the impending symptoms.

The seizure itself is the ictus, providing another opportunity for administration of a dose of medication to minimize the severity and/or duration of the seizure. At this stage, the administration of the medication may be accomplished by a person within the patient's environment.

### Prodromal Symptoms

The prodromal group of symptoms occurs days or hours before a seizure ensues, providing a reasonable warning period of an impending seizure which, in turn, provides a reasonable opportunity to begin administration of an appropriate medication to prevent the seizure and/or minimize the duration and time of the seizure.

The prodromal phase includes symptoms that a patient can distinguish from the symptoms of the aura stage, e.g.:

### Affection;

### Depression;

Difficulty concentrating;
Ecstatic feelings;

### Headache;

Increased agitation;

### Insomnia;

### Irritability;

### Lightheadedness; and

Mood changes.

### Auras

Auras are actually a small partial seizure that is often followed by a larger event, usually coming a few seconds to a few minutes before the actual seizure and, therefore, provide a very short warning period that requires rapid action in administering a medication to prevent the seizure and/or minimize the effects of the seizure such as its duration and/or severity of the seizure itself. Further, because the time of the warning period is so short, it is highly desirable that the administered medication reach the patient's CNS as quickly as possible to begin therapeutic action.

The symptoms in the Aura phase include:

Biting of tongue (from teeth clenching when muscles tighten);
Blinking of eyes, eyes may move to one side or look upward, or staring;
Convulsion (person loses consciousness, body becomes rigid or tense, then fast jerking movements occur);
Change in skin color (looks pale or flushed);
Difficulty talking (may stop talking, make nonsense or garbled sounds, keep talking or speech may not make sense);
Difficulty breathing;
Dreamlike experiences;
Dressing or undressing;

### Drooling;

Feelings of extreme fear;

### Hallucinations;

Heart racing;
Inability to swallow;
Lack of movement or muscle tone (unable to move, loss of tone in neck and head may drop forward, loss of muscle tone in body and person may slump or fall forward);
Losing control of urine or stool unexpectedly;
Movements of hands, like wringing, playing with buttons or objects in hands;
Non-purposeful movements, or automatisms, involve the face, arms or legs, such as Lip smacking or chewing movements;
Pupils may dilate or appear larger than normal;
Repeated purposeful movements (person may continue activity that was going on before the seizure);
Rigid or tense muscles in part of the body or the whole body;
Sensations in the stomach;

### Sweating;

Tremors, twitching or jerking movements (may occur on one or both sides of face, arms, legs or whole body; may start in one area then spread to other areas or stay in one place);
Unpleasant smells; and
Walking or running.

### Ictal Phase

The middle of a seizure is called the ictal phase and is the period of time from the first symptoms (including an aura) to the end of the seizure activity, which correlates with the electrical seizure activity in the brain and represents another opportunity for a care-giver or individual within the environment of the patient to administer one or more doses of medication to minimize the severity of the ictal phase of the seizure. It would be highly desirable to provide an easy administration mechanism that is non-invasive and that is as fast-acting as possible within the patient's CNS.

Common symptoms during a seizure include:
Awareness, sensory, emotional or thought change;
Biting of tongue (from teeth clenching when muscles tighten);
Blinking of eyes, eyes may move to one side or look upward, or staring;
Body parts feels or looks different;
Blurry vision;
Change in skin color (looks pale or flushed);
Confused, feeling spacey;
Convulsion—person loses consciousness, body becomes rigid or tense, then fast jerking movements occur;
Déjà vu (feeling of being there before but never have);
Difficulty breathing;
Difficulty talking (may stop talking, make nonsense or garbled sounds, keep talking or speech may not make sense);
Distracted and/or daydreaming;
Dressing or undressing;
Euphoric feelings;
Feeling detached;
Feeling of panic, fear, impending doom—intense feeling that something bad is going to happen;
Hashing lights;
Formed visual hallucinations;
Heart racing;
Inability to hear;
Inability to swallow, drooling;
Jamais vu (feeling that something is very familiar but it isn't);
Lack of movement or muscle tone (unable to move, loss of tone in neck and head may drop forward);
Loss of awareness (often called “black out”);
Loss of consciousness, unconscious, or “pass out”;
Losing control of urine or stool unexpectedly;
Loss of muscle tone in body—person may slump or fall forward;
Loss of vision or inability to see;
Movements of hands, like wringing, playing with buttons or objects in hands;
Non-purposeful movements, or automatisms, involve the face, arms or legs, such as lip smacking or chewing movements;
Numbness, tingling, or electric shock like feeling in body, arm or leg;
Out of body sensations;
Periods of forgetfulness or memory lapses;
Pupils may dilate or appear larger than normal;
Repeated purposeful movements (person may continue activity that was going on before the seizure);
Rigid or tense muscles in part of the body or the whole body;
Sounds may be strange or different;

### Sweating;

Tremors, twitching or jerking movements (may occur on one or both sides of face, arms, legs or whole body; may start in one area then spread to other areas or stay in one place);
Unusual smells may be sensed;
Unusual tastes; and
Walking or running.

### Postictal Stage

The postictal stage occurs after the ictus or active stage of the seizure and represents the recovery period after the seizure. Some patients recover immediately, while others may take minutes to hours to feel normal. The type of seizure, as well as what part of the brain the seizure impacts, affects the recovery period—how long it may last and what may occur during it.

Common symptoms after a seizure include:

### Anxiety;

### Confusion;

Depression, sadness, upset;
Difficulty talking or writing;

### Embarrassment;

Emotional behavior;

### Exhaustion;

### Fear;

Feeling fuzzy, light headed or dizzy;

### Frustration;

General weakness or weak in one part or side of the body;

### Headache;

Lack of awareness;
Memory loss;
Nausea or upset stomach;
Physical changes;
Sensory changes;

### Shame;

### Sleepiness;

Slow response or an inability to respond;

### Thirst;

Thought changes;
Urge to go to the bathroom or lose control of bowel or bladder;
They also may suffer such injuries as:
Broken bones;

### Bruising;

### Cuts;

Head injuries;

Patients diagnosed with epilepsy or seizure disorders do not presently have a rapid and safe way to either stop, or mitigate the effects of, a sensed impending seizure. It would be very desirable, therefore, to provide a treatment method that may be used as a very fast-acting preventive method, to prevent an impending sensed seizure. Moreover, the treatment method should be able to be administered quickly and easily by the patient when a seizure is sensed and by a care-giver or other person during the seizure itself to help mitigate or minimize the effects of the seizure, including severity and duration.

The present invention provides solutions for, inter alia, these problems.

### SUMMARY OF THE INVENTION

The present system is directed in one embodiment to a method of administration of a therapeutic compound and/or composition for patients with epilepsy or seizure disorders. The method includes administering one or more therapeutic compositions comprising an effective amount of insulin directly to the subject patient's CNS, with no, or minimal, systemic exposure. Preferably, this method comprises administration to the upper third of a patient's nasal cavity, thereby bypassing the patient's blood-brain barrier and delivering the therapeutic compound and/or composition directly to the patient's central nervous system.

### DETAILED DESCRIPTION OF THE INVENTION

### Definitions

As used herein, “central nervous system” (CNS) refers to the brain and spinal cord and associated cells and tissues.

As used herein, “systemic administration” refers to administration of a medication, pharmaceutical and the like by the following non-limited means: oral, intravenous, intra-arterial, intramuscular, epidermal, transdermal, subcutaneous, topic, sublingual as well as general inhalation, for example, administration to the lower two-thirds of the patient's nasal cavity or other method with delivery to the patient's lungs and non-CNS circulatory system. In each of these cases, the administered drug will migrate through the patient's circulatory system and, in order to reach the patient's CNS would be required to cross the patient's blood-brain barrier.

In the context of the present invention, the terms “treat” and “therapy” and the like refer to alleviate, slow the progression of, prophylaxis, attenuation and/or minimization of the seizure including duration and/or severity, the condition causing seizures within the CNS.

“Prevent”, as used herein, refers to putting off, delaying, slowing, inhibiting, or otherwise stopping, reducing or ameliorating the onset of seizures. It is preferred that a large enough quantity of the therapeutic agent(s) and/or compound(s) be applied in non-toxic levels in order to provide an effective level of activity against the impending, or ongoing seizure. The method of the present invention may be used with any animal, such as a mammal or a bird (avian), more preferably a mammal. Poultry are a preferred bird. Exemplary mammals include, but are not limited to rats, mice, cats, dogs, horses, cows, sheep, pigs, and more preferably humans.

An “effective amount” of therapeutic agent(s), i.e., insulin, and/or component(s) of the pharmaceutical composition of the present invention comprising therapeutic agent(s) is an amount sufficient to treat, reduce and/or ameliorate the symptoms, neuronal damage and/or underlying causes of epilepsy or seizure disorder. Preferably, at least an effective amount of the at least one therapeutic agent, i.e., insulin, and/or component(s) of the pharmaceutical composition yields a tissue concentration in the range of about 10⁻¹³ molar to about 10⁻⁸ molar, but the concentrations may be greater provided that toxicity is avoided. Generally, at least an effective amount of insulin or pharmaceutical composition(s) thereof is administered in order to ensure that an effective amount of insulin is delivered to the target CNS for treating epilepsy or seizure disorders.

The concentration range of insulin delivered to the upper third of the patient's nasal cavity may be preferably in the range of 10⁻⁹ molar to about 10⁻³ molar in order to yield the preferable tissue concentration range of about 10⁻¹³ molar to about 10⁻⁸ molar, though as discussed above, concentrations in the tissue may be higher so long as toxicity is avoided.

For illustrative purposes only, exemplary treatment regimens relating generally to the therapeutic agent, i.e., insulin, and/or pharmaceutical compounds disclosed herein, including dosage ranges, volumes and frequency are provided below:

Efficacious dosage range for the at least one therapeutic agent, i.e., insulin, delivery-enhancement agents, vasoconstrictors and/or antibiotics comprises 1×10⁻⁷ to 0.1 mg/kg.
A more preferred dosage range may be 1×10⁻⁴ to 0.1 mg/kg.
The most preferred dosage range may be 0.01 to 1 mg/kg.
The dosage volume (applicable to nasal sprays or drops) range may be 0.015 ml-1.0 ml.
The preferred dosage volume (applicable to nasal sprays or drops) range may be 0.03 ml-0.6 ml.

The brain concentrations that are likely to be achieved with the dosage ranges provided above are for each of the therapeutic agents described above, including insulin, for a single dose: 1×10⁻¹³ to 1×10⁻⁸ M. Over the course of a multi-dose treatment plan, the maximum brain concentration may be as high as 50 μM for delivery-enhancement agents and antibiotics.

The present disclosure is generally directed to administering insulin intranasally to patients for treatment of epilepsy and/or seizure disorders.

Generally, the method of the present invention comprises treating a patient with epilepsy or seizure disorders with the direct non-invasive delivery of a therapeutic, i.e., effective, amount or dose of insulin, or a pharmaceutical composition thereof, to the CNS. This may be accomplished by administration of at least an effective or therapeutic amount of insulin, or pharmaceutical composition thereof, to the upper one-third of the patient's nasal cavity, thereby delivering the effective or therapeutic amount or dose directly to the patient's CNS, with minimal systemic exposure. Multiple clinical trials have been conducted using insulin delivered as described herein, i.e., to the upper third of the nasal cavity, each such clinical study reporting no clinically significant change in the blood levels of either insulin or glucose.

In some embodiments, the therapeutic agent—insulin—may be combined with a vasoconstrictor to be administered intranasally to limit systemic exposure. The vasoconstrictor may be administered to the nasal cavity prior to administration of the therapeutic compound to the upper third, or alternatively to the lower two-thirds, of the nasal cavity or, still more alternatively, the vasoconstrictor and therapeutic compound may be administered concurrently, either to the upper one-third or the lower two-thirds of the patient's nasal cavity. Thus, the present invention allows for a safe and efficacious treatment of a patient's epilepsy and/or seizure disorders where systemic administration or exposure is contraindicated.

In addition, patients with epilepsy and/or seizure disorders typically have blood (systemic) insulin and glucose levels that are within the normal range, unless the disease is severe and advanced. Systemic administration in these patients will, as discussed herein, result in loss of the administered insulin to non-target systemic insulin receptors, organs and tissues, presenting an increased risk of insulin shock or catastrophic hypoglycemia as blood insulin levels rise and blood glucose levels fall. A relatively small portion of the systemically administered insulin will cross the blood-brain barrier to reach the patient's CNS tissues, but in order to achieve an “effective dose” the insulin dosing will require a massive and dangerous overdosing to compensate for the systemic loss, highlighting the risk of insulin shock or catastrophic hypoglycemia in patients with epilepsy or seizure disorders. As a result, it would not be obvious to treat patients with epilepsy or seizure disorders with insulin.

However, even if the particular patient with epilepsy or seizure disorders has abnormal glucose levels, treatment of insulin with the presently claimed administration to the upper third of the patient's nasal cavity with direct delivery to the CNS will have very little effect on the patient's blood insulin and glucose levels. This is because, once the delivered insulin dosing is located within the patient's CNS, very little, if any, of the insulin within the CNS will be transported out of the CNS to the patient's bloodstream; the transport of insulin into the CNS is far faster and much more efficient than transport of insulin out of the CNS. In this way, the dangers of systemic exposure of insulin in patients with epilepsy or seizure disorders is avoided in two respects: first, minimization of systemic exposure by directly delivering an effective amount of insulin directly to the brain; and second, once the delivered effective amount of insulin is within the brain, the vast majority of the insulin delivered directly to the brain/CNS remains within the brain/CNS. Consequently, even patients with abnormal glucose levels may be safely, and very rapidly, treated using the present invention.

Moreover, the various embodiments of the present invention are very fast-acting since the at least an effective amount of insulin is delivered directly to the patient's CNS by bypassing the patient's blood-brain barrier. This is critical in the case of patients with epilepsy and/or seizure disorders with an impending, or ongoing, seizure.

Certain embodiments may comprise methods for reducing the severity of seizures in a patient, comprising:
administering at least an effective amount of insulin to the upper third of the nasal cavity of the patient before and/ or during the seizure;
enabling at least an effective amount of insulin to directly access the patient's central nervous system by bypassing the blood-brain barrier; and
thereby reducing the severity of the seizures in the patient.

Other embodiments may comprise methods for treating refractory epilepsy and/or seizures in a patient comprising: administering at least an effective amount of insulin to the upper third of the nasal cavity of the patient on a daily, BID, TID or other ongoing basis;
enabling at least an effective amount of insulin to directly access the patient's central nervous system by bypassing the blood-brain barrier; and
thereby reducing the frequency of the seizures, the severity of the seizures and/or the duration of the seizures in the patient.

Thus, the present invention may not only treat or prevent seizures, but the number of seizures and their severity may also be reduced.

Certain embodiments of the present invention are, therefore, directed to a non-invasive and fast-acting medicament that is delivered directly to the patient's CNS either on a regular basis or treatment regimen, e.g., daily, TID, BID, and/or just prior to the onset of a seizure and/or during a seizure that is ongoing. The use of the present invention in this manner provides a reduction in the likelihood that the seizure will begin and/or the severity of the seizure itself. In turn, this reduces the risk of a patient dying from a condition known as Sudden Unexpected Death in Epilepsy (“SUDEP”) which may also be caused by a lack of brain insulin signaling.

It is known that doctors recommend the ketogenic diet for children whose seizures have not responded to seizure medicines. The ketogenic diet comprises a special high-fat, low-carbohydrate diet that helps to control seizures in some people. Most typically, the ketogenic diet, also called the “long-chain triglyceride diet,” provides 3 to 4 grams of fat for every gram of carbohydrate and protein. The ketogenic diet, therefore, is a way to provide energy to brain cells when normal insulin stimulated glucose uptake into the brain and its metabolism is not providing sufficient energy. It has been demonstrated that the ketogenic diet does reduce or prevent seizures in children whose seizures could not be controlled by conventional medications.

There are a variety of types of insulin available that are suitable for use in accordance with the present disclosure, including insulins for which zinc is included for stabilization and others which do not include zinc. Because zinc may be detrimental to the olfactory system, insulins that do not contain zinc may be preferable in some cases. Formulations of insulin that either contain no preservatives (which could be prepared for unit dosing) or a safe preservative such as pyrophosphate are preferred. In some embodiments the insulin formulation may not include any phenol or cresol preservatives.

Moreover, different forms of insulin may be used with the present invention. For example, and without limitation, “modified insulins”, “chemically modified insulins”, and “post-translationally modified insulins” as described in J. L. Dunne et al., Diabetes 61:1907-1914, 2012, which is hereby incorporated by reference in its entirety.

Some additional exemplary insulin forms that may be administered according to the various embodiments of the present invention include:
Insulin analogs such as insulin Lispro rDNA, insulin aspart, Glulisine (Apidra) which is a zinc-free insulin, insulin detemir, Insulin degludec, Insulin glargine,
Iodination of a conserved tyrosine (TyrB26) enhances key properties of a rapid-acting clinical analog of insulin as described by Krystel E l Hage, et al., Extending Halogen-based Medicinal Chemistry to Proteins. Journal of Biological Chemistry, 2016; 291 (53): 27023 DOI: 10.1074/jbc.M116.761015, the entire contents of which are hereby incorporated by reference.
NPH insulin.
Animal insulins (may have a different amino acid sequence from human insulin).
Ultra-rapid acting, short acting, intermediate acting, and long-acting insulins.

The insulin, or composition comprising insulin, may be administered intranasally to the upper third of the patient's nasal cavity as a powder, spray, gel, ointment, infusion, injection, or drops, for example. The insulin may be administered in an effective dose. The insulin or insulin composition may also be dispensed as a powder or liquid nasal spray, nose drops, a gel or ointment, through a tube or catheter, by syringe, by packtail, by pledget, or by submucosal infusion. Any suitable nasal spray device may be used with embodiments of the present disclosure.

An at least an effective amount, as herein defined, of the therapeutic compound, i.e., insulin, to be administered pursuant to embodiments of the invention is the most preferred method of expression of dosage. Such effective amount is dependent upon many factors, including but not limited to, the type of disease or condition and its severity, i.e., the severity, including the duration, of the patient's seizures, the patient's general health, size, age, and the nature of the treatment, i.e. short-term or chronic treatment.

Generally, the treatment may be given in a single dose or multiple administrations, i.e., once, twice, three or more times daily over a period of time, e.g., BID, TID or other regular basis. In some cases, one or more doses daily may be given over an extended period of time, including, months or years. In some cases, the treatment may be self-administered, or administered by a care-giver, when one or more of the sensations described above are experienced and sensed by the patient. These may be given over an extended period of time to help prevent the seizure and/or minimize the severity thereof. Alternatively, or in addition, the treatment may be administered during the very short warning period of the Aura. Further, the treatment may be administered by a care giver during the actual seizure period to help minimize the severity of the seizure. Finally, the treatment may be administered post-seizure to help shorten the recovery phase.

The method of the invention administers an at least an effective amount of the insulin, or pharmaceutical compound thereof, to the upper third of the nasal cavity of a mammal. It is preferred that the at least an effective amount of insulin be administered to the olfactory area in the upper third of the nasal cavity and particularly to the olfactory epithelium in order to promote transport of the agent into the peripheral olfactory neurons rather than the capillaries within the respiratory epithelium. In some embodiments it may be preferable to transport insulin to the brain by means of the nervous system instead of the circulatory system so that therapeutic agents that are unable to cross the blood-brain barrier from the bloodstream into the brain may be delivered to damaged neurons in the brain.

### Transportation Pathway to Bypass Blood-Brain Barrier

### The Olfactory Nerve

Various methods of the present invention include administration of at least an effective amount of insulin and/or pharmaceutical composition(s) thereof to tissue innervated by the olfactory nerve and that is located in the upper third of the nasal cavity. The at least an effective amount of insulin and/or pharmaceutical composition(s) thereof can be delivered to the olfactory area via application to the upper third of the nasal cavity.

Fibers of the olfactory nerve are unmyelinated axons of olfactory receptor cells that are located in the upper one-third of the nasal mucosa. The olfactory receptor cells are bipolar neurons with swellings covered by hair-like cilia that project into the nasal cavity. At the other end, axons from these cells collect into aggregates and enter the cranial cavity at the roof of the nose. Surrounded by a thin tube of pia, the olfactory nerves cross the subarachnoid space containing CSF and enter the inferior aspects of the olfactory bulbs. Once the therapeutic agent(s) and/or pharmaceutical composition(s) of the present invention is applied to the upper third of nasal cavity, the therapeutic agent(s) and/or pharmaceutical composition(s) of the present invention can undergo transport through the nasal mucosa and into the olfactory bulb and other areas of the CNS, such as the anterior olfactory nucleus, frontal cortex, hippocampal formation, amygdaloid nuclei, nucleus basalis of Meynert, hypothalamus, midbrain, cerebellum, cervical spinal cord and the like.

### Neuronal Transport

Embodiments of the present method includes administration of an at least an effective amount of insulin and/or pharmaceutical composition(s) thereof of the present invention to the subject by application to the upper third of the mammalian subject's nasal cavity. Application of the at least an effective amount of insulin and/or pharmaceutical composition(s) thereof of the present invention in this manner ensures that an effective amount of insulin and/or pharmaceutical composition(s) are transported to the CNS, brain, and/or spinal cord along a neural pathway, with reduced systemic loss and, therefore, minimized systemic exposure. A neural pathway includes transport within or along a neuron, through or by way of lymphatics running with a neuron, through or by way of a perivascular space of a blood vessel running with a neuron or neural pathway, through or by way of an adventitia of a blood vessel running with a neuron or neural pathway, or through an hemangiolymphatic system.

The present invention comprises transportation of the administered insulin and/or pharmaceutical composition(s) thereof by way of a neural pathway, rather than through the circulatory system, so that agent(s) and/or compound(s) that are unable to, or only poorly, cross the blood-brain barrier from the bloodstream into the brain can be delivered to the lymphatic system, CNS, brain, and/or spinal cord. The therapeutic agent(s) and/or pharmaceutical composition(s) of the present invention, once past the blood-brain barrier and in the CNS, can then be delivered to various areas of the brain or spinal cord through lymphatic channels, through a perivascular space, or transport through or along neurons.

Use of a neural pathway to transport a therapeutic agent(s) and/or pharmaceutical composition(s) to the brain, spinal cord, or other components of the central nervous system obviates the obstacle presented by the blood-brain barrier so that medications, i.e., therapeutic agent(s) and/or pharmaceutical compositions of the present invention, that cannot normally cross that barrier, can be delivered directly to the CNS, e.g., the brain and spinal cord. In addition, the present invention can provide for delivery of a more concentrated level of the therapeutic agent(s) and/or pharmaceutical composition(s) of the present invention to the CNS since the therapeutic agent(s) and/or pharmaceutical composition(s) of the present invention do not become diluted in fluids present in the bloodstream. As such, the invention provides an improved method for delivering an effective amount or therapeutic dose of the administered insulin and/or pharmaceutical composition(s) thereof directly to the target CNS including the brain and/or spinal cord.

### The Olfactory Neural Pathway

One embodiment of the present method includes delivery of the effective amount of insulin to the subject's CNS for prevention and treatment of epilepsy and/or seizure disorders in a manner such that the at least an effective amount of insulin administered to the upper third of the nasal cavity is transported into the CNS, e.g., the brain, and/or spinal cord along an olfactory neural pathway. Typically, such an embodiment includes administering the at least an effective amount of insulin and/or other compound(s) to tissue innervated by the olfactory nerve and inside the nasal cavity. The olfactory neural pathway innervates primarily the olfactory epithelium in the upper third of the nasal cavity, as described above. Application of the at least an effective amount of insulin to a tissue innervated by the olfactory nerve can deliver an effective amount of insulin and/or compound(s) to damaged neurons or cells of the CNS, including but not limited to the brain, and/or spinal cord. Olfactory neurons innervate this tissue and can provide a direct connection to the CNS, brain, and/or spinal cord due, it is believed, to their role in olfaction.

Delivery through the olfactory neural pathway can employ lymphatics that travel with the olfactory nerve to the various brain areas and from there into dural lymphatics associated with portions of the CNS, such as the spinal cord. Transport along the olfactory nerve can also deliver an effective amount of insulin and/or composition thereof to an olfactory bulb. A perivascular pathway and/or a hemangiolymphatic pathway, such as lymphatic channels running within the adventitia of cerebral blood vessels, can provide an additional mechanism for transport of an effective amount of insulin, e.g., to the brain and spinal cord from tissue innervated by the olfactory nerve.

At least an effective amount of insulin, and/or pharmaceutical compositions thereof may be administered to the olfactory nerve, for example, through the olfactory epithelium located at the upper one-third of the nasal cavity. Such administration can employ extracellular or intracellular (e.g., transneuronal) anterograde and retrograde transport of the agent(s) and/or compound(s) entering through the olfactory nerves to the brain and its meninges, to the brain stem, or to the spinal cord. Once the at least an effective amount, i.e., therapeutic dose, of the insulin and/or pharmaceutical composition thereof is dispensed into or onto tissue innervated by the olfactory nerve, the administered insulin and/or pharmaceutical composition and/or components thereof may be transported through the tissue and travel along olfactory neurons into areas of the CNS including but not limited to the brain stem, cerebellum, spinal cord, cerebrospinal fluid, olfactory bulb, and cortical and subcortical structures. Thus, an effective amount of insulin and/or pharmaceutical composition thereof, is delivered to the target CNS for prevention and/or treatment of epilepsy and/or seizure disorder.

The blood-brain barrier is bypassed in the present invention by application of at least an effective amount of insulin and/or pharmaceutical composition(s) comprising insulin and/or composition(s) or compound(s) to the upper third of the nasal cavity of the patient, e.g., a mammal. The administered amount of the insulin and/or pharmaceutical composition thereof of the invention migrate from the nasal mucosa through foramina in the cribriform plate along the olfactory neural pathway and an effective amount is delivered directly into the CNS. Administration to the nasal cavity employing a neural pathway can thus deliver an effective amount of therapeutic agent(s), e.g., insulin and/or pharmaceutical compositions thereof to the lymphatic system, brain stem, cerebellum, spinal cord, and cortical and subcortical structures of the mammalian patient. The therapeutic agent(s), e.g., insulin and/or pharmaceutical composition(s) thereof of the present invention alone may facilitate this movement into the CNS, i.e., brain, and/or spinal cord. Alternatively, a carrier and/or the delivery-enhancement agent(s) may assist in the transport of the administered insulin and/or pharmaceutical composition of the present invention into and along the neural pathway. Administration of the insulin and/or pharmaceutical composition(s) thereof of the present invention to the upper third of the mammalian patient's nasal cavity thus enables the administered therapeutic agent(s) to bypass the blood-brain barrier through a transport system from the nasal mucosa and/or epithelium to the CNS, i.e., brain and spinal cord where an effective amount of the administered insulin is delivered.

Various embodiments of the invention administer an at least an effective amount of insulin and/or pharmaceutical composition(s) thereof of the present invention to tissue innervated by the olfactory nerves. Such nerve systems can provide a direct connection between the outside environment and the brain, thus providing advantageous delivery of the agent(s) and/or compound(s) to the CNS, including brain, brain stem, and/or spinal cord. The administered insulin and/or pharmaceutical composition(s) thereof of the present invention may be unable to cross or inefficiently cross the blood-brain barrier from the bloodstream into the brain. Alternatively, for those agent(s) and/or composition(s) that may cross the blood-brain barrier, the present invention offers an alternative treatment for those patients having a concurrent system, non-CNS disease or condition that contraindicates systemic administration of the therapeutic agent(s) and/or compositions(s) needed within the CNS to treat a first CNS-related disease, condition or disorder. Thus, the methods of the present invention allow for the delivery of an effective amount of insulin and/or pharmaceutical composition(s) thereof to the target CNS by way of the olfactory nerve rather than through the circulatory system in order to facilitate prevention and/or treatment of epilepsy and/or seizure disorders. Thus, this method of administration of at least an effective amount of insulin to the upper third of the nasal cavity and delivery of the effective amount of insulin to the target CNS allows for the efficient and non-invasive delivery of an effective amount of insulin and/or pharmaceutical composition(s) thereof of the present invention to the CNS, brain, or spinal cord without systemic loss or exposure.

### Alternative Pathways

Alternative non-systemic pathways to the olfactory nerve pathway discussed above comprise pathways along other nerves that innervate the nasal cavity, e.g., the trigeminal pathway, well known to the skilled artisan.

Administration of Therapeutic Agent(s) and/or Pharmaceutical Compounds

Administering insulin according to the methods of the invention for treatment of epilepsy and/or seizure disorders may include application of at least an effective amount of the therapeutic agent, i.e., insulin alone or formulating the at least an effective amount of insulin with at least an effective amount of one or more of the compounds described supra as pharmaceutical compositions and administering the pharmaceutical compositions to a mammalian subject or host, including a human patient, intranasally to the upper third of the nasal cavity. The therapeutic agent(s) and/other components of the pharmaceutical composition thereof, e.g., vasoconstrictor, delivery-enhancement agent and/or antibiotic may be administered at one of a variety of doses sufficient to provide an effective amount at the desired point of action in the CNS for the administered at least an effective amount of insulin and/or pharmaceutical composition component.

When included, vasoconstrictor(s), delivery-enhancement agent(s), and/or antibiotic(s) may be delivered as pre-treatment, co-treatment and/or post-treatment with the therapeutic agent(s) and/or pharmaceutical composition, either alone or as a component of the pharmaceutical composition. Delivery of at least an effective amount of insulin in this manner results in delivery of an effective amount of insulin to the target CNS with maximum efficiency in the delivery of insulin, i.e., with minimal to no systemic exposure of insulin.

For application to the upper third of the nasal cavity as suspensions, aerosols, sprays or drops, the at least an effective amount of insulin and/or pharmaceutical composition(s) can be prepared according to techniques well known in the art of pharmaceutical formulation. The compositions can be prepared as suspensions of the agent(s) in solutions which may comprise salts such as saline, components such as phosphate, succinate or citrate buffers to maintain pH, osmoregulatory and osmotic agents such as taurine, and suitable preservatives, absorption promoters to enhance bioavailability, fluorocarbons or other solubilizing or dispersing agents known in the art. The means of applying a pharmaceutical composition intranasally to the upper third of the nasal cavity may be in a variety of forms such as a powder, spray, gel or nose drops.

Other forms of compositions for administration of the at least an effective amount of insulin and/or pharmaceutical compositions or elements thereof include a suspension of a particulate, such as an emulsion, a liposome, or in a sustained-release form to prolong the presence of the pharmaceutically active agent in an individual. The powder or granular forms of the pharmaceutical composition may be combined with a solution and with a diluting, dispersing or surface-active agent. Additional compositions for administration include a bioadhesive to retain the agent at the site of administration at the upper third of the nasal cavity, for example a spray, paint, or swab applied to the mucosa. A bioadhesive can refer to hydrophilic polymers, natural or synthetic, which, by the hydrophilic designation, can be either water soluble or swellable and which are compatible with the pharmaceutical composition. Such adhesives function for adhering the formulations to the mucosal tissues of the upper third of the nasal cavity. Such adhesives can include, but are not limited to, hydroxypropyl cellulose, hydroxypropyl methylcellulose, hydroxy ethylcellulose, ethylcellulose, carboxymethyl cellulose, dextran, gaur gum, polyvinyl pyrrolidone, pectins, starches, gelatin, casein, acrylic acid polymers, polymers of acrylic acid esters, acrylic acid copolymers, vinyl polymers, vinyl copolymers, polymers of vinyl alcohols, alkoxy polymers, polyethylene oxide polymers, polyethers, and combinations thereof. The composition can also be in the form of lyophilized powder, which can be converted into solution, suspension, or emulsion before administration. The pharmaceutical composition is preferably sterilized by membrane filtration and is stored in unit-dose or multi-dose containers such as sealed vials or ampoules.

The pharmaceutical composition may be formulated in a sustained-release form to prolong the presence of the active therapeutic agent(s) in the treated individual. Many methods of preparation of a sustained-release formulation are known in the art and are disclosed in Remington's Pharmaceutical Sciences. Generally, the therapeutic agent(s), pharmaceutical composition and/or components of the pharmaceutical composition, i.e., vasoconstrictor, delivery-enhancement agent and/or antibiotic may be entrapped in semi-permeable matrices of solid hydrophobic polymers. The matrices can be shaped into films or microcapsules. Matrices can include, but are not limited to, polyesters, co-polymers of L-glutamic acid and gamma ethyl-L-glutamate, polylactides, polylactate polyglycolate, hydrogels, non-degradable ethylene-vinyl acetate, degradable lactic acid-glycolic acid copolymers, hyaluronic acid gels, and alginic acid suspensions. Suitable microcapsules can also include hydroxymethylcellulose or gelatin and poly-methyl methacrylate. Microemulsions or colloidal drug delivery systems such as liposomes and albumin microspheres can also be used.

### Delivery Systems

The therapeutic agent, i.e., insulin, and/or a pharmaceutical composition comprising at least an effective dose of insulin and/or components of the pharmaceutical composition of the present invention may further be dispensed and applied to the upper third of the nasal cavity as a powdered or liquid nasal spray, suspension, nose drops, a gel, film or ointment, through a tube or catheter, by syringe, by packtail, by pledget (a small flat absorbent pad), by nasal tampon or by submucosal infusion. In some aspects of the present invention, the methods comprise administering to an individual the at least an effective dose of insulin and/or a pharmaceutical composition thereof to the upper third of the nasal cavity by way of a delivery device. Nasal drug delivery can be carried out using devices including, but not limited to, nasal spray devices, unit dose containers, pump sprays, droppers, squeeze bottles, airless and preservative-free sprays, nebulizers (devices used to change liquid medication to an aerosol particulate form), metered dose inhalers, and pressurized metered dose inhalers. In some aspects, an accurate effective dosage amount is contained within a bioadhesive patch that is placed directly within and on the upper third of a nasal cavity.

At least an effective dose of insulin and/or a pharmaceutical composition comprising at least an effective dose of insulin and/or components of the therapeutic composition of the present invention may be conveniently delivered to the upper third of the nasal cavity in the form of an intranasal aerosol spray using a pressurized pack or a nebulizer and a suitable propellant including, but not limited to: dichlorodifluoromethane, trichlorofluoromethane, dichlorotetrafluoroethane, hydrocarbons, compressed air, nitrogen or carbon dioxide. An aerosol system requires the propellant to be inert towards the therapeutic agent(s) and/or pharmaceutical composition as will be readily recognized by the skilled artisan. In the case of a pressurized aerosol, the dosage unit may be controlled by providing a valve to deliver an accurately metered amount. Nasal spray devices that are designed to target the upper third of the nasal cavity are preferred.

The means to deliver the at least an effective amount of insulin or pharmaceutical composition comprising the at least an effective amount of insulin and/or components of the pharmaceutical composition of the present invention to the upper third of the nasal cavity as a powder may be in a form such as microspheres delivered by a nasal insufflator device (a device to blow a gas, powder, or vapor into a cavity of the body) or pressurized aerosol canister. The insufflator produces a finely divided cloud of the dry powder or microspheres. The insufflator may be provided with means to ensure administration of a substantially metered amount of the pharmaceutical composition. The powder or microspheres should be administered in a dry, air-dispensable form. The powder or microspheres may be used directly with an insufflator which is provided with a bottle or container for the powder or microspheres. Alternatively the powder or microspheres may be filled into a capsule such as a gelatin capsule, or other single dose device adapted for nasal administration. The insufflator can have means such as a needle to break open the capsule or other device to provide holes through which jets of the powdery composition can be delivered to the upper third of the nasal cavity.

### Intermittent and Cyclic Dosing

In various embodiments of the invention, therapeutic agent, i.e., insulin, and/or a pharmaceutical composition comprising at least an effective amount of insulin and/or the components of the pharmaceutical composition may be administered as a single and one-time dose, or alternatively the at least an effective amount of insulin and/or the components of the pharmaceutical composition may be administered more than once and intermittently. By “intermittent administration” is intended administration of at least an effective amount of insulin and/or the components of the pharmaceutical composition, followed by a time period of discontinuance, which is then followed by another administration of the at least effective amount, and so forth. Administration of the at least an effective amount of insulin and/or the components of the pharmaceutical composition may be achieved in a continuous manner, as for example with a sustained-release formulation, or it may be achieved according to a desired daily dosage regimen, as for example with one, two, three, or more administrations per day. By “time period of discontinuance” is intended a discontinuing of the continuous sustained-released or daily administration of the insulin and/or the components of the pharmaceutical composition. The time period of discontinuance may be longer or shorter than the period of continuous sustained-release or daily administration. During the time period of discontinuance, the concentration(s) of insulin and/or the components of the pharmaceutical composition level in the relevant tissue is substantially below the maximum level obtained during the treatment. The preferred length of the discontinuance period depends on the concentration of the effective dose and the form of therapeutic agent(s) and/or the components of the pharmaceutical composition used. The discontinuance period can be at least 2 days, preferably is at least 4 days, more preferably is at least 1 week and generally does not exceed a period of 4 weeks. When a sustained-release formulation is used, the discontinuance period must be extended to account for the greater residence time of the at least one therapeutic agent. Alternatively, the frequency of administration of the effective dose of the sustained-release formulation can be decreased accordingly. An intermittent schedule of administration of insulin and/or the components of the pharmaceutical composition may continue until the desired therapeutic effect, and ultimately treatment of the disease or disorder is achieved.

In yet another embodiment, intermittent administration of the at least an effective amount(s) of insulin and/or the components of the pharmaceutical composition is cyclic. The term “cyclic” is intended herein to refer to intermittent administration accompanied by breaks in the administration, with cycles ranging from about 1 month to about 2, 3, 4, 5, or 6 months. For example, the administration schedule might be intermittent administration of the at least an effective dose of insulin and/or the components of the pharmaceutical composition, wherein a single short-term dose is given once per week for 4 weeks, followed by a break in intermittent administration for a period of 3 months, followed by intermittent administration by administration of a single short-term dose given once per week for 4 weeks, followed by a break in intermittent administration for a period of 3 months, and so forth. As another example, a single short-term dose may be given once per week for 2 weeks, followed by a break in intermittent administration for a period of 1 month, followed by a single short-term dose given once per week for 2 weeks, followed by a break in intermittent administration for a period of 1 month, and so forth. A cyclic intermittent schedule of administration of insulin and/or the components of the pharmaceutical composition to a subject may continue until the desired therapeutic effect, and ultimately treatment of the disorder or disease is achieved.

The invention has been described with reference to various specific and preferred embodiments and techniques. However, it should be understood that many variations and modifications may be made while remaining within the spirit and scope of the invention.

We claim:

1. A method for preventing and/or treating seizures in a patient, comprising:
administering at least an effective amount of insulin to the upper third of the nasal cavity of the patient;
enabling at least an effective amount of insulin to directly access the patient's central nervous system by bypassing the blood-brain barrier; and
preventing and/or treating the patient's seizures.

2. The method of claim 1, further comprising administering the insulin to a tissue innervated by the olfactory nerve and/or the trigeminal nerve, wherein the administered insulin bypasses the blood-brain barrier to access the patient's central nervous system to treat the patient's seizures.

3. The method of claim 1, further comprising administering the at least an effective amount of insulin to the patient while the patient is experiencing a seizure.

4. The method of claim 1, further comprising administering the at least an effective amount of insulin to the patient after conclusion of a seizure.

5. The method of claim 4, further comprising administering the at least an effective amount of insulin to the patient while the patient is experiencing a seizure.

6. The method of claim 1, wherein the at least an effective amount of insulin is in the range of 1×10⁻⁷ to 0.1 mg/kg.

7. The method of claim 6, wherein a more preferred dosage range for the at least an effective amount of insulin is in the range of 1×10⁻⁴ to 0.1 mg/kg.

8. The method of claim 1, wherein the concentration of insulin in the brain of the patient after a single dose in in the range of 1×10⁻¹³ to 1×10⁻⁸ molar.

9. A method for treating and/or preventing a seizure in a patient who senses an impending seizure, comprising:
determining that one or more warning signs of an impending seizure are sensed by the patient;
administering at least an effective amount of insulin to the upper third of the nasal cavity of the patient before the sensed impending seizure is actually experienced by the patient;
enabling at least an effective amount of insulin to directly access the patient's central nervous system by bypassing the blood-brain barrier; and
thereby treating and/or preventing the impending seizure.

10. The method of claim 9, further comprising the administering of the at least an effective amount of insulin to the patient during the actual experiencing of the seizure by the patient.

11. The method of claim 9, further comprising the administering of the at least an effective amount of insulin to the patient after the patient experiences the sensed seizure.

12. The method of claim 10, further comprising the administering of the at least an effective amount of insulin to the patient after the patient experiences the sensed seizure.

13. The method of claim 9, wherein the at least an effective amount of insulin is in the range of 1×10⁻⁷ to 0.1 mg/kg.

14. The method of claim 9, wherein the concentration of insulin in the brain of the patient after a single dose in in the range of 1×10⁻¹³ to 1×10⁻⁸ molar.

15. A method for rapid rescue of a patient experiencing a seizure, comprising:
administering at least an effective amount of insulin to the upper third of the nasal cavity of the patient during and/or after the seizure;
enabling at least an effective amount of insulin to directly access the patient's central nervous system by bypassing the blood-brain barrier;
minimizing the severity and/or duration of the seizure; and
thereby rapidly rescuing the patient from the seizure.

16. The method of claim 15, wherein the at least an effective amount of insulin is in the range of 1×10⁻⁷ to 0.1 mg/kg.

17. The method of claim 15, wherein the concentration of insulin in the brain of the patient after a single dose in in the range of 1×10⁻¹³ to 1×10⁻⁸ molar.

18. A method for shortening the recovery time for a patient after the patient experiences a seizure, comprising:
administering at least an effective amount of insulin to the upper third of the nasal cavity of the patient during and/or after the seizure;
enabling at least an effective amount of insulin to directly access the patient's central nervous system by bypassing the blood-brain barrier; and
thereby shortening the recovery time of the patient after experiencing the seizure.

19. The method of claim 18, wherein the at least an effective amount of insulin is in the range of 1×10⁻⁷ to 0.1 mg/kg.

20. The method of claim 18, wherein the concentration of insulin in the brain of the patient after a single dose in in the range of 1×10⁻¹³ to 1×10⁻⁸ molar.

21. A method for preventing and/or treating sudden unexpected death in epilepsy in patients with epilepsy or other seizure disorders, comprising:
administering at least an effective amount of insulin to the upper third of the nasal cavity of the patient before a sensed impending seizure and/or during the seizure and/or after the seizure;
enabling at least an effective amount of insulin to directly access the patient's central nervous system by bypassing the blood-brain barrier; and
preventing the sudden unexpected death in epilepsy and/or treating the patient for sudden unexpected death in epilepsy.

22. The method of claim 21, wherein the at least an effective amount of insulin is in the range of 1×10⁻⁷ to 0.1 mg/kg.

23. The method of claim 21, wherein the concentration of insulin in the brain of the patient after a single dose in in the range of 1×10⁻¹³ to 1×10⁻⁸ molar.

24. A method for reducing the frequency and/or duration of a seizure in a patient, comprising:
administering at least an effective amount of insulin to the upper third of the nasal cavity of the patient during the experiencing of a seizure by the patient;
enabling at least an effective amount of insulin to directly access the patient's central nervous system by bypassing the blood-brain barrier; and
thereby reducing the frequency of the seizure and/or the duration of the seizure experienced by the patient.

25. The method of claim 24, further comprising administering the at least an effective amount of insulin after the seizure has concluded.

26. The method of claim 24, wherein the at least an effective amount of insulin is in the range of 1×10⁻⁷ to 0.1 mg/kg.

27. The method of claim 24, wherein the concentration of insulin in the brain of the patient after a single dose in in the range of 1×10⁻¹³ to 1×10⁻⁸ molar.

28. A method for reducing the severity of seizures experienced by a patient, comprising:
administering at least an effective amount of insulin to the upper third of the nasal cavity of the patient during the seizure;
enabling at least an effective amount of insulin to directly access the patient's central nervous system by bypassing the blood-brain barrier; and
thereby reducing the severity of the seizures experienced by the patient.

29. The method of claim 28, further comprising the patient sensing an oncoming seizure by recognizing warning signs of the oncoming seizure; and administering the at least an effective amount of the insulin to the patient before the patient experiences the oncoming seizure.

30. The method of claim 29, wherein the at least an effective amount of insulin is in the range of 1×10⁻⁷ to 0.1 mg/kg.

31. The method of claim 29, wherein the concentration of insulin in the brain of the patient after a single dose in in the range of 1×10⁻¹³ to 1×10⁻⁸ molar.

32. A method for treating refractory epilepsy and/or seizures in a patient comprising:
administering at least an effective amount of insulin to the upper third of the nasal cavity of the patient according to a regular established treatment regimen;
enabling at least an effective amount of insulin to directly access the patient's central nervous system by bypassing the blood-brain barrier; and
thereby reducing the frequency of the seizures, the severity of the seizures and/or the duration of the seizures in the patient.

33. The method of claim 32, further comprising administering the at least an effective amount of insulin before, during and/or after a seizure.
